SEQUENCE LISTING (3 sequences)

SEQ ID NO: 1 - DNA, 33 bp, Homo sapiens
aagatggatgcagaattccgacatgactcagga

SEQ ID NO: 2 - DNA, 54 bp, Homo sapiens
ttaatgatgatgatgatgatgcgctatgacaacaccgcccaccatgagtccaat

SEQ ID NO: 3 - protein, 42 aa, Homo sapiens [sequence omitted]

(19) United States
(12) Patent Application Publication
(10) Pub. No.: US 2005/0043264 A1
(43) Pub. Date: Feb. 24, 2005

(54) Methods of inhibiting neurodegenerative disease

(76) Inventors: Jyh-Lyh Juang, Taipei City (TW); Ho Lin, Taipei City (TW)

Correspondence Address: Finnegan, Henderson, Farabow,;Garrett & Dunner, L.L.P., 1300 I Street, N.W., Washington, DC, 20005-3315, US

(21) Appl. No.: 10/883,150

(22) Filed: Jul. 1, 2004

Related U.S. Application Data
(60) Provisional application No. 60/484,094, filed on Jul. 1, 2003.

(51) Int. Cl. A61K 48/00; A61K 38/48

(52) U.S. Cl. 514/44; 514/2

(57) ABSTRACT

The invention provides methods for treating neurodegenerative disease as well as methods for screening compounds for the treatment of neurodegenerative disease. Transgenic animals, e.g., insects, which may be used as a model for neurodegenerative disease, are also provided.

### DESCRIPTION OF THE INVENTION

1. Field of the Invention

The invention relates generally to the field of neurodegenerative diseases, e.g., Alheimer's disease. In certain embodiments, the invention relates to methods of treating neurodegenerative diseases. Other embodiments of the invention relate to methods of screening for compounds which inhibit neurodegenerative diseases. Certain embodiments of the invention provide a transgenic animal model for neurodegenerative diseases.

2. Background of the Invention

Alheimer's disease (AD) is the most frequently diagnosed neurodegenerative disease, and is characterized histologically by two distinguishing hallmarks: senile plaques, which are extracellular deposits of β-amyloid (Aβ₄₂), and intracellular neurofibrillary tangles of aggregated hyperphosphorylated tau protein (Giannakopoulos et al.,1996, J. Neuropathol. Exp. Neurol. 55:1210). It has been shown that the accumulation of Aβ₄₂ precedes the pathological changes associated with neurodegeneration (Pike et al.,1993, J. Neurosci. 13:1676) and that inhibition of Aβ₄₂ accumulation can effectively intervene in the AD pathogenesis (Cherny et al., 2001, Neuron 30: 665). Less clear is the contribution of the downstream protein targets of Aβ₄₂toxicity in neurons. Understanding the Aβ₄₂-induced signaling cascade may provide clues for designing therapeutic strategies for treatment of AD.

One component of the signaling pathway associated with AD pathogenesis is cyclin dependent kinase 5 (Cdk5). Cdk5 is activated by Aβ₄₂, the protein found in AD associated plaques (Alvarez et al., 2001, Exp. Cell Res. 264:266). Deregulated Cdk5 is correlated with tau hyperphosphorylation which leads to one hallmark of AD associated pathology (Noble et al., 2003, Neuron 38:555). Cdk5 requires association with its regulatory partners for kinase activation. p35 is one known activating partner, and is expressed primarily in postmitotic neurons (Nikolic et al., 1996, Genes Dev. 10:816; Tsai et al., 1994, Nature 371:419). While under normal physiological conditions, Cdk5 associates with p35 in healthy neurons, Cdk5 appears to be deregulated by its association with p25, a calpain digestion product of p35 found in AD neurons (Patrick et al., 1999, J. Biol. Chem. 273:24057). It has been hypothesized that the complex of Cdk5/p25 hyperphosphorylates tau to reduce its association with microtubules, which subsequently results in neuronal apoptosis (Zhang et al., 2002, J. Neurochem. 81:307). Neuronal apoptosis is yet another hallmark associated with AD pathology (Glabe, 2001, J. Mol. Neurosci. 17:137; Yankner, 1996, Neuron 16:921).

Abl, another cellular kinase, is a nonreceptor tyrosine kinase distributed both in the nucleus and cytoplasm of proliferating cells. Abl kinase appears to be evolutionarily conserved from fly, e.g., Drosophila melanogaster, to human, functioning in the developing nervous system (Hoffmann, 1991, Trends Genet. 7:351; Van Etten, 1999, Trends Cell Biol. 9:179). Evidence suggests that Abl kinase participates in the regulation of apoptosis (Barila et al, 2003, Mol. Cell Biol. 23:2790; Wang, 2000, Oncogene 19:5643). Abl has not, however, previously been associated with AD pathogenesis.

While the molecular mechanisms underlying neurodegenerative diseases have been widely investigated, therapeutic targets for the treatment of neurodegenerative diseases such as AD still must be identified. Screening systems to evaluate therapeutic agents that can treat neurodegenerative diseases are also needed.

### SUMMARY OF THE INVENTION

The invention relates, in part, to the discovery that Abl kinase (Abl) actively participates in the pathogenesis of neurodegenerative diseases such as AD. Inhibiting Abl abrogates both activation of Cdk5 and its translocation in neurons expressing or containing Aβ₄₂, thus inhibiting neurodegeneration associated with AD.

Embodiments of the invention, provide methods of treating a subject having a neurodegenerative disease, such as, e.g., AD, comprising administering to the subject, an agent which inhibits Abl kinase activity.

Certain embodiments of the invention provide methods of inhibiting degeneration of a neuron comprising contacting the neuron with an agent which inhibits Abl kinase activity. In some embodiments the degeneration is apoptotic.

The invention also provides methods of inhibiting formation of a complex comprising Abl, Cdk5, Cables, Abi and p35 or a fragment thereof in a neuron comprising contacting the neuron with an agent which inhibits Abl kinase activity.

The invention further provides methods of inhibiting Cdk5 activity in a cell comprising contacting the cell with an agent which inhibits Abl kinase activity thereby inhibiting Cdk5 activity.

Certain embodiments of the invention provide methods of screening for an agent which treats a neurodegenerative disease comprising contacting a cell with the agent and measuring Abl kinase activity in the cell where a decrease in Abl kinase activity in the cell contacted with the agent compared to a cell not contacted with the agent indicates that the agent can be used to treat a neurodegenerative disease. In certain embodiments the neurodegenerative disease is AD.

In some embodiments the invention provides a method of screening for an agent which inhibits degeneration of a neuron comprising contacting a cell with the agent and measuring the Abl kinase activity in the cell, where a decrease in Abl kinase activity in the cell contacted with the agent compared to a cell not contacted with the agent indicates that the agent can be used to treat neurodegeneration.

In some embodiments, the invention provides a transgenic animal, including a transgenic insect such as Drosophila melanogaster, comprising a transgene comprising a nucleotide sequence encoding a β amyloid protein, e.g., Aβ₄₂.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 shows the suppression of Abl kinase activity and that Abl expression prevents Aβ₄₂-induced neuronal death in Drosophila. FIG. 1A is an immunoprecipitation and Western blot showing kinase activity in BG2-c2 cells treated with Aβ₄₂ and/or STI571 (Abl kinase inhibitor). BG2-c2 cells were treated with Aβ₄₂ (2 μM) and/or an Abl kinase inhibitor (STI571, 20 μM) for one hour to investigate the effect on overexpressed-Ena phosphorylation by Abl. FIG. 1B shows that Aβ₄₂-induced neuronal death was suppressed by STI571 in Drosophila neuronal cells. Neuronal viability was determined by MTT assays. FIG. 1C shows that Abl protein affected Aβ₄₂-induced neuronal death. dsRNA-abl was introduced by transfection two days before treating with Aβ₄₂ for 24 hours. Viability values of control group were adjusted to 100% (n=4); **: p<0.01 versus control group; ++: p<0.01 versus the Aβ₄₂ group. FIG. 1D shows controls of dsRNA-abl. The efficiency and specificity of dsRNA-abl (0˜20 μg/2×10⁶ cells) were evaluated by monitoring the expression of HA tagged-Abl, Cdk5, and actin (upper panel). dsRNA-EGFP was used as non-specific control (lower panel).

FIG. 2A shows a co-immunoprecipitation and Western blot of Cdk5 and Abl. Abl-Cdk5 interaction was enhanced by activation of Abl activity. Neuronal cells were transfected with Abl-HA 2 days before treatment with Aβ₄₂ (2 μM) or STI571 (20 μM) for one hour. Cell lysates were subjected to anti-HA immunoprecipitation followed by both anti-Cdk5 and anti-HA immunoblotting. FIG. 2B shows an immunoprecipitation and Western blot of Cdk5 and Abl (top panel) and the result of an in vitro kinase assay using the immunoprecipitates and histone H1 as a substrate (bottom panel). Elevation of Cdk5 kinase activity required Abl kinase activity. The numbers below the bottom panel indicate the relative intensities of histone H1 phosphorylation.

FIG. 3 shows the effects of ectopic expression of Aβ₄₂ in Drosophila eye. Ectopic expression of Aβ₄₂ induces neurodegeneration in eye imaginal disc and adult rough eye phenotypes, which are suppressed in Abl mutant flies. FIG. 3A shows Aβ₄₂ was ectopically expressed by the eye-specific gmr-GAL4 driver in wild-type and Abl mutant flies. Apoptotic cells in eye discs of 3rd-instar larvae were detected by TUNEL assay as shown in FIG. 3A, panel b. FIG. 3B shows adult eye phenotypes examined by scanning electron microscope (SEM). Panels e, f, g, and h are enlarged from relative positions in panels a, b, c, and d, respectively. Note the defects of bristles and facets in Aβ₄₂-expression compound eyes. Scale bar: 0.1 mm.

FIG. 4A shows an immunoprecipitate and Western blot of Cdk5 and phophorylated Cdk5 (Cdk5-pY15)(top panel) and the correlation with kinase activity in an in vitro assay using H1 histone as a substrate over time (bottom panel). Drosophila neuronal cells were treated with Aβ42 (2 μM) for 15-60 minutes to investigate its effect on Cdk5-pY15 and Cdk5 kinase activity over the time-course. FIG. 4B shows an immunoprecipitation and a Western blot of Cdk5 and phophorylated Cdk5 (Cdk5-pY15)(top panel) and the correlation with kinase activity in an in vitro assay using H1 histone as a substrate (bottom panel) with and without the Abl kinase inhibitor STI571. Inhibition of Abl kinase reduced Aβ₄₂-induced Cdk5-pY15 and Cdk5 kinase activity. STI571 (20 μM) and/or A(342 (2 μM) were added simultaneously to neuronal cells for 60 minutes. FIG. 4C is similar to FIG. 4B except that STI571 has been replaced with double stranded RNA-abl (dsRNA-abl) or double stranded RNA-EGFP (dsRNA-EGFP) as a non specific control. Suppression of Abl protein expression with ds-RNA-abl reduced Aβ4₂-induced Cdk5 kinase activity. Cdk5-pY15 was markedly suppressed (though not entirely) by transfection of dsRNA-abl into BG2-c2 cells (20 μg/2×10⁶ cells). Comparable levels of immunoprecipitated Cdk5 were detected with anti-Cdk5 antibody. FIG. 4D shows an immunoprecipitation and a Western blot of Cdk5 and phosphorylated Cdk5 (Cdk5-pY15)(top panel) and the correlation with kinase activity in an in vitro assay using H1 histone as a substrate (bottom panel) from lysates derived from the heads of transgenic flies expressing wild type Abl or abl mutants. Cdk5 protein from different strains of fly heads were examined for Y15 phosphorylation and kinase activity. The numbers below the figures represent the relative Cdk5 kinase activity. GMR is a control fly not expressing Aβ₄₂ (lane 1); GMR>Aβ₄₂ overexpresses Aβ₄₂ in a wild type Abl background (lane 2); Abl mutant is not overexpressing Aβ₄₂ (lane 3); and GMR>Aβ₄₂Abl mutant overexpresses Aβ₄₂ in the presence of the Abl mutant (lane 4).

FIG. 5A schematically shows the structural conservation of p35 between human and Drosophila. The putative calpain digesting site is shown in human p35 sequence, but the consensus sequence is not conserved in Drosophila (highlighted characters show the conserved amino acids). MS, represents the myristoylation site. CRD represents the Cdk5 regulatory domain. FIG. 5(B) shows that Drosophila p35 is not cleaved to p25 in the presence of Aβ₄₂ either in vitro (left panel) or in vivo (right panel). Left panel, two days after transfection of HA-tagged Dp35, Drosophila neuronal cells were treated with 5 or 10 μM Aβ₄₂ or calcium ionophore (A23187 with 5 μM CaCl2) for an additional 24 hours. Cell lysates were immunoblotted with anti-HA and anti-actin antibodies to examine the Dp35 cleavage and protein loading respectively. Right panel, adult fly heads ectopically expressing Aβ₄₂ and/or Dp35-HA (driven by gmr-GAL4) were lysed and immunoblotted with anti-HA and anti-actin antibodies. Consistently, these results revealed no detectable cleavage or reduction of Dp35-HA protein (˜53 kDa) in either the cell culture or transgenic systems.

FIG. 6A (left panel) is a Western blot showing that expression of exogenous Dp35-HA is suppressed in a dose dependant manner by double stranded RNA-Dp35 (dsRNA-DP35). The right panel shows that dsRNA-Dp35 suppresses kinase activity of Cdk5 in an in vitro kinase assay using histone H1 as a substrate, but does not effect Cdk5 phosphorylation (Cdk5-pY15) in response to Aβ₄₂. Y15 phosphorylation level and kinase activity of Cdk5 were determined while Dp35 was targeted by dsRNA in cells treated with Aβ₄₂. dsRNA-Dp35 was transfected into neuronal cells two days before Aβ₄₂ treatment. After one hour of Aβ₄₂ treatment, cells were lysed and immunoprecipitated with anti-Cdk5 antibody to examine Cdk5-pY15 and kinase activity. The Cdk5-pY15 signal was largely retained but its kinase activity was dramatically abolished. FIG. 6B shows the results of a TUNEL assay of apoptotic cells in eye discs of 3rd-instar larvae. Apoptosis in Drosophila eye discs caused by the Dp35-transgene was mitigated in the Abl mutant background. Scale bar: 0.1mm. FIG. 6C are SEM images of adult transgenic fly compound eye phenotypes corresponding respectively to the results in 6B.

FIG. 7 shows that Aβ₄₂ affects Cdk5 intraneuronal translocalization by a p25-independent, Abl-associated phosphorylation of Cdk5-Y15 mechanism in Drosophila neuronal cells. FIG. 7A shows confocal microscopy analysis of immunostaining of endogenous Cdk5 (TRITC-labeled) in BG2-c2 neuronal cells. Cdk5 translocalized from the cell membranes (a) to perinuclear regions (b) following stimulation of cells with 2 μM Aβ₄₂ for one hour. Nuclei were labeled with DAPI (blue) (b). Cdk5 translocalization was blocked by simultaneously adding 5 μM BL-1 (a Cdk5 kinase inhibitor) with Aβ₄₂ to suppressed Cdk5 kinase activation by Aβ₄₂ (c). Aβ₄₂-stimulated Cdk5 translocalization to perinuclear cytoplasm reverted back to the plasma membrane by inhibiting Cdk5 kinase activity one hour after Aβ₄₂ treatment (d). FIG. 7B shows that phosphorylation of Cdk5-Y15 is essential for Aβ₄₂-induced Cdk5 translocalization. BG2-c2 cells were cotransfected with an EGFP plasmid (panels g and h) and with Cdk5-Y15F mutant to differentiate the mutant Cdk5-expressing cells (panel e and arrow in panel f) from the non-transfected cells. Stimulation of cells with Aβ₄₂ for one hour markedly translocalized endogenous wild-type Cdk5 protein to the perinuclear regions (arrowhead in panel f) in contrast to that of exogenous Cdk5-Y15F protein (arrow in panel f). FIG. 7C shows that Abl kinase activity is important for Aβ₄₂-stimulated Cdk5 translocalization. Cdk5 and Abl-HA were double immunolabeled to investigate whether they colocalized upon Aβ₄₂ stimulus. Without treatment of Aβ₄₂, Abl-HA (FITC-labeled) and Cdk5 (TRITC-labeled) were colocalized to the cell membrane and cytoplasm (panels i, l, and o). As expected, Abl-HA and Cdk5 colocalized to perinuclear regions in response to Aβ₄₂ stimulus (panels j, m, and p). White arrow indicates an Abl-HA transfected cell exhibited no Abl or Cdk5 protein translocalization to perinuclear region when cells were simultaneously treated with STI571 and Aβ₄₂ (panels k, n, and q). Scale bar: 10 μm.

FIG. 8 shows that Abl functions cooperatively with p25 and active Cdk5 in Aβ₄₂-induced human neuronal death. FIG. 8A shows that STI571 effectively suppressed Aβ₄₂-induced IMR-32 neuronal death after 72 hours-treatment with Aβ₄₂. Cells were pretreated for 24 hours with STI571 before stimulation with Aβ₄₂ for 24 hours or 72 hours (n=4, 24-hour control value is considered 100%; **: p<0.01 versus control group in the same time point; ++: p<0.01 versus Aβ₄₂ group). Induction of p35 cleavage into p25 by Aβ₄₂ is incapable of initiating Cdk5 kinase activity in cells lacking Abl kinase activity. Human IMR-32 cells were pretreated for 24 hours with STI571 before stimulation with/without Aβ₄₂ (20 μM) for 24 hours to examine the effect on Cdk5-pY15 and Cdk5 kinase activity. FIG. 8B shows that the cleavage of p35 into p25 by the induction of Aβ₄₂ was not noticeably affected by STI571, but Cdk5 kinase activity and Y15 phosphorylation was suppressed by STI571 treatment. FIG. 8C shows that co-treatment of calpain inhibitor (Calpeptin) and STI571 further reduced Cdk5 activity compared to individual treatment (lane 5 vs. lane 3 and 4). The numbers below the figures represent the relative Cdk5 kinase activity. FIG. 8D is a schematic diagram of the Aβ₄₂-triggered neurodegeneration model showing the role for Abl in deregulation of Cdk5 kinase activity and subcellular localization.

### DESCRIPTION OF THE EMBODIMENTS

The invention is based, in part, on the discovery that Abl kinase (Abl) actively participates in the pathogenesis of neurodegenerative diseases, such as, e.g., AD. As a result of this discovery, methods of treating neurodegenerative diseases can be developed based on inhibition of Abl which reduces neurodegeneration associated with diseases such as AD.

A. Definitions

“Amyloid precursor protein” (APP), as used herein, refers to a heterogeneous group of ubiquitously expressed polypeptides migrating between 110 and 140 kDa on electrophoretic gels. This heterogeneity arises both from alternative splicing (yielding 3 major isoforms of 695, 751, and 770 residues) as well as by a variety of posttranslational modifications, including the addition of N- and O-linked sugars, sulfation, and phosphorylation. The APP splice forms containing 751 or 770 amino acids are widely expressed in nonneuronal cells throughout the body and also occur in neurons. Neurons express higher levels of the 695-residue isoform. APP is the precursor of Aβ₄₂, associated with AD plaques. The cleavage of amyloid precursor protein by three different protease activities termed α-, β-, and γ-secretase is the decisive event by which either the pathogenic amyloid β (Aβ₄₂) peptides associated with AD or the harmless (and possibly even beneficial) APPsα fragments are released. In humans the gene encoding APP is found on chromosome 21.

“Aβ₄₂,” as used herein, refers to a cleavage product of APP comprising (SEQ ID NO: 3). In some cases Gly may be substituted for Ala at position 21 of SEQ ID NO: 3. In some cases Gin may be substituted for Glu at position 22 of SEQ ID NO: 3. In some cases Val may be substituted for Ala at position 42 of SEQ ID NO: 3.

“Abl,” as used herein, refers to a non-membrane bound tyrosine kinase found in a variety of cell types including neurons. In one activated form, Bcr-Abl, it is oncogenic causing various forms of leukemia in humans. Phosphorylation of tyrosine is one activity associated with Abl and the specific inhibition of this activity with STI571 is used to treat leukemia. Abl is described in greater detail infra.

“Alheimer's disease,” (AD) as used herein, refers to a progressive degenerative disease of the brain that causes impairment of memory and dementia manifested by confusion, visual-spatial disorientation, inability to calculate and deterioration of judgment. Atrophy of the cerebral cortex, with consequent enlargement of sulci and ventricles may be grossly evident on imaging studies. Histologically the cortex, hippocampus and amygdala show atrophy of neurons, with cytoplasmic vacuoles and argentophillic granules; distortion of intracellular neurofibrils (neurofibrillary tangles) due to excessive phosphorylation of microtubular tau proteins; and plaques composed of granular or filamentous argentophillic masses with a core of the 42 amino acid form of β amyloid (Aβ₄₂). The concentration of tau protein in the cerebrospinal fluid is increased, while the concentration of Aβ₄₂ is decreased.

“Cdk5,” as used herein, refers to cyclin dependent protein kinase 5. Cdk5 is a serine/threonine kinase which requires p35 (or p25) binding for activation. Cdk5 plays a role in neuritic outgrowth, cortical lamination and synaptogenesis.

“Degeneration of a neuron,” as used herein, refers to the cell death of a neuron, either in vivo or in vitro. The cell death may, in some cases, be the result of apoptosis.

“Erbstatin analog,” as used herein, refers to non-specific inhibitors of tyrosine kinase activity in epidermal growth factor (EGF) and Abl. One example is 2,5-dihydroxymethylcinnamate which is commercially available.

“Kinase,” as used herein, refers to an enzyme which can catalyze the transfer of phosphate groups usually to a tyrosine, threonine or serine residue. Protein phosphorylation is a basic mechanism for modification of protein function in eukaryotic cells. Phosphorylation and dephophorylation of proteins is a chemical signaling mechanism used by cells to relay messages from the outside environment (i.e., outside the cell membrane) to the interior of the cell. Typically the signal is ultimately transferred to the nucleus where it alters gene expression. Tyrosine kinases are involved in immune, endocrine and nervous system physiology and pathology.

“Nucleic acid,” as used herein, refers to polymers comprised of deoxyribonucleotides or ribonucleotides in either single- or double-stranded form. The term “nucleic acid” encompasses nucleic acids containing naturally occurring nucleotides as well as analogues of natural nucleotides that have binding properties similar to the reference nucleic acid. The term nucleic acid also includes cDNA or an mRNA encoded by a gene. A nucleic acid will be able to hybridize to its complement through complementary base pairing, e.g., via a hydrogen bond.

An “oligonucleotide,” as used herein, refers to a single-stranded nucleic acid that typically is less than or equal to 100 bases long. Of course, complementary oligonucleotides may be annealed to form double-stranded nucleic acids. As used herein, an oligonucleotide may include natural (i.e., A, G, C, T, or U) or modified bases. In addition, the bases in an oligonucleotide may be joined by a linkage other than a phosphodiester bond, so long as it does not interfere with interstrand base pairing. Thus, for example, oligonucleotides may be peptide-nucleic acids in which the constituent bases are joined by peptide bonds rather than phosphodiester linkages (see, e.g., Nielson, 2001, Current Opinion in Biotechnology 12:16). Optionally, the oligonucleotides may be directly labeled with a detectable substance such as radioisotopes, chromophores, lumiphores, chromogens, or ECL moieties or may be indirectly labeled, for example, with biotin to which a streptavidin or avidin complex may later bind.

“Neurodegenerative disease,” as used herein, refers to any condition characterized by the progressive loss of neurons, due to cell death, in the central nervous system of a subject.

“Polypeptide”, as used herein, refers to a polymer of amino acids and does not refer to a specific length of the product; thus, peptides, oligopeptides, and proteins are included within the definition of polypeptide. This term does not exclude post-expression modifications of the polypeptide, for example, glycosylation, acetylation, phosphorylation, pegylation, addition of a lipid moiety, or the addition of any organic or inorganic molecule. Included within the definition, are for example, polypeptides containing one or more analogs of an amino acid (including, for example, unnatural amino acids) and polypeptides with substituted linkages, as well as other modifications known in the art, both naturally occurring and non-naturally occurring.

“Specific binding partner,” as used herein, refers to a first molecule that can form a relatively stable complex with a second molecule under physiologic conditions. In general, specific binding is characterized by a relatively high affinity and a relatively low to moderate capacity. Nonspecific binding usually has a low affinity with a moderate to high capacity. Typically, binding is considered specific when the affinity constant Kₐ is higher than about 10⁶M⁻¹, or is higher than about 10⁸M⁻¹. A higher affinity constant indicates greater affinity, and thus greater specificity. Antibodies typically bind antigens with an affinity constant in the range of 10⁶M⁻¹ to 10⁹M⁻¹ or higher. If desired, nonspecific binding may be reduced without substantially affecting specific binding by varying the binding conditions. Such conditions are known in the art, and a skilled artisan using routine techniques can select appropriate conditions. The conditions may be defined, for example, in terms of molecular concentration, ionic strength of the solution, temperature, time allowed for binding, concentration of unrelated molecules (e.g., serum albumin, milk casein), etc.

“Subject”, as used herein, means an animal or an insect including a human or non-human mammal, e.g., a dog, a cat, a mouse, a rat, a cow, a sheep, a pig, a goat, a non-human primate, or an insect, such as a fly.

“Transgene,” as used herein, refers a gene engineered by the human hand to be expressed in an organism such as unicellular organism, e.g., a prokaryotic or eukaryotic cell, or a multicellular organism such as a plant, a fungus or an animal, e.g., a mammal, an insect.

“Treat,” “treatment,” and “treating,” as used herein refer to any of the following: the reduction in severity of a disease or condition; the reduction in the duration of a disease course; the amelioration of one or more symptoms associated with a disease or condition; the provision of beneficial effects to a subject with a disease or condition, without necessarily curing the disease or condition; the prophylaxis of one or more symptoms associated with a disease or condition.

B. Pathological Events in Alheimer's Disease

Two events are associated with the AD pathology. The first is the accumulation of β amyloid, e.g., Aβ₄₂, plaques in the brain. The second is the hyper-phosphorylation of the protein tau.

Aβ₄₂ is a cleavage product of APP. APP is first cleaved by α or β secretase generating a C terminal fragment (C₉₉ or C₈₃) which is then cleaved by γ secretase (presenilin complex) to generate β amyloid (Aβ₄₂ or Aβ₄₀). Aβ₄₂ is more hydrophobic and thus more apt to aggregate into plaques. The Aβ₄₂ isoform is considered to be the pathogenic agent in AD.

Tau hyperphosphorylation has been implicated in altering the association of tau and cellular microtubule proteins leading to cytoskeletal abnormalities (Lee et al., 2000, Nature 405:360). It is believed this disruption leads to apoptotic neuronal cell death. Aβ₄₂ activates the serine/threonine protein kinase Cdk5. Cdk5 deregulation has been implicated in tau hyperphophorylation. Cdk5 association with p25, a calpain cleavage product of cellular protein p35, is believed to contribute to the aberrant activation and translocation of Cdk5 from the cell membrane to the perinuclear cytoplasm. These events are associated with tau hyperphosphorylation and AD pathogenesis.

The invention is based in part on the discovery that the uncleaved p35 protein can associate with and activate Cdk5. Interestingly, p35 has been demonstrated to be resistant to calpain-mediated cleavage after being phosphorylated by Cdk5 in the developing rat brain (Saito et al., 2003, J. Neurosci. 23:1189). It has been suggested that Cdk5 in accompany with p35 could be transported into nucleus in primary cortical neurons to facilitate apoptosis (Gong et al., 2003, Neuron 38:33; Weishaupt et al., 2003, Cell Tissue Res. 312:1). Cdk5-p25 is a more stable complex with a longer half-life that prolongs the activation of Cdk5 (Patrick et al., 1999, Nature 402:615). The invention is further based in part on the discovery that the formation of p25 does not sufficiently trigger Cdk5 activity when Abl activation is blocked, indicating a critical role for Abl in the neurodegenerative process.

C. Abl Kinase

Abl is a nonreceptor protein tyrosine kinase found in the inner leaflet of the plasma membrane, cytosol, endosomal membranes and nucleus. Abl kinase activity and subcellular localization are tightly regulated in normal physiology. Deregulation of Abl kinase has been implicated in several diseases, for example, the Bcr-Abl fusion oncoprotein is the disease hallmark of the chronic myelogenous leukemia (CML). The Bcr-Abl fusion protein results from a chromosomal translocation and plays a causative role in certain human leukemias. Abl kinase has also been implicated in the regulation of apoptosis (Wang, 2000, Oncogene 19:5463; Barila et al., 2002, Mol. Cell. Biol. 23:2790). Abl has been reported to modulate F-actin cytoskeleton and neurite extension (Woodring et al., 2002, J. Cell Biol. 156:879). Abl is activated in response to oxidative stress (Sun et al., 2000, J. Biol. Chem. 275:17237). The nuclear form of Abl is positively regulated by genotoxic stress, such as DNA damage (Kharbanda et al., 1995, J. Biol. Chem. 270:30278).

One substrate of Abl is the cellular protein Ena. Together, they play a role in cytoskeletal regulation during cell-cell adhesion. Ena regulates actin dynamics and cell motility of fibroblasts.

Reports suggest that Abl activates Cdk5 during brain development by phosphorylating tyrosine-15 (Y15) on Cdk5. Abl activation of Cdk5 is mediated by the adaptor protein Cables (Zukerberg et al., 2000, Neuron 26:633). Another important adaptor protein is Abi. Cables can form a tri-complex with Abl and Cdk5. Complex formation can result in initiation of Cdk5 kinase activity. Abi is considered as a kinase substrate and a positive kinase regulator of Abl. Thus adaptor proteins of Abl may be implicated in the regulation of Cdk5 kinase activity in AD pathogenesis.

It has now been discovered that Abl may form a complex comprising Cdk5 and p35 or a fragment thereof, e.g., p25, in neurons expressing or containing Aβ₄₂. Abl may also contribute to the aberrant activation of Cdk5 and may cause Cdk5 to translocate from the membrane to the perinuclear region in neurons expressing or containing Aβ₄₂. The triggering of each of these events is associated with the pathogenesis of AD.

This invention demonstrates for the first time that Abl is essential for Aβ₄₂-triggered neurodegeneration both in vivo and in vitro and that Abl can serve as a molecular therapeutic target to stop the progress of AD pathogenesis. The anti-leukemic agent Abl kinase inhibitor, STI571, originally designed to suppress oncogenic Abl kinase activity in chronic myelogenous leukemia is shown herein to prevent the Aβ₄₂-induced neurodegeneration in both Drosophila and mammalian cells.

D. Abl Inhibitors

An Abl inhibitor may be any agent which inhibits at least one activity associated with Abl involved in a cellular signaling pathway leading to neurodegeneration in vivo, i.e., in a subject, or degeneration of a neuron in vitro. An activity associated with Abl may include the phosphorylation of a substrate. The substrate may be a polypeptide such as another kinase or alternatively the substrate may be Abl itself.

Upon activation, some kinases translocate within the cell. As an example, Cdk5 translocates from the cell membrane to the perinuclear cytoplasm, in the presence of Aβ₄₂. The translocation is believed to be associated with neurodegeneration seen in AD. Moreover, inhibiting Abl can inhibit the downstream translocation of Cdk5. Thus, in some embodiments the Abl inhibitor may inhibit cellular translocation of a kinase capable of participating in neurodegeneration, such as Cdk5.

Kinases may bind to specific binding partners within a cell to form complexes comprised of intracellular signaling molecules. As an example, in the presence of Aβ₄₂, a complex comprising Abl, p35, Cdk5 may form and lead to neurodegeneration. Adaptor proteins, e.g., Abi and Cables which bind Abl can be a part of the complex. In certain cell types, e.g., human neurons, calpain may cleave p35 to form p25 thereby allowing Abl, p25, and cdk5 to form a more stable complex resulting in neurodegenration. In certain embodiments the invention provides Abl inhibitors which prevent complex formation and thereby inactivate a kinase, e.g., Abl, or Cdk5. Inhibitors can include specific binding partners of any of the proteins which form the complex, e.g., Abl, p35, Cdk5, Abi, and Cables.

Any type of molecule may be used as an Abl inhibitor. The molecule may be a polypeptide, e.g., a specific binding partner. The molecule may be a small organic or inorganic molecule, e.g., imatinib mesylate, i.e., STI571, 6-(2,6-dichlorophenyl)-8-methyl-2-(3-methylsulfanylphenyl-amino)-8H-pyridol[2,3-d]pyrimidin-7-one, or an erbstatin analog. In certain embodiments the small molecule, e.g., STI571, may be modified, made more lipophollic, formulated as a nanoparticle, to facilitate penetration of the blood brain barrier. The molecule may be a nucleic acid, e.g., DNA, RNA or an oligonucleotide comprised of DNA or RNA. The nucleic acid may be single stranded or double stranded. The nucleic acid may hybridize to cellular DNA or RNA encoding a kinase, e.g., Abl and thereby inhibit transcription or translation of the gene encoding the kinase.

In some embodiments an Abl inhibitor may be an analog of a specific binding partner of Abl. An analog of a specific binding partner may include specific binding partners that have been chemically modified so that they retain their binding activity but prevent at least one of phosphorylation of a substrate, translocation of a kinase such as Cdk5, or formation of a complex comprised of Abl. In other embodiments a specific binding partner may be used as an Abl inhibitor, e.g., a compound which specifically binds to Abl and inhibits at least one activity associated with Abl.

In some embodiments the agent may be a double stranded RNA molecule. In specific embodiments the double stranded RNA molecule may encode Abl. The RNA molecule can encode a full length Abi protein or a fragment of an Abl protein so long as it inhibits the cellular expression of Abl when it is administered in vivo to a subject or in vitro to a cell.

In one specific embodiment, the Abl inhibitor is a nucleic acid that can be used in RNA interference (RNAi ), e.g., short hairpin RNA (shRNA). RNAi is an evolutionarily conserved phenomenon in which gene expression is suppressed by the introduction of homologous double-stranded RNAs (dsRNAs). After dsRNAs are delivered to the cytoplasm of the cell, they are cleaved by the enzyme Dicer to 21-23 nucleotide small interfering RNAs. These siRNAs are then incorporated into a protein complex, the RNA-induced silence complex (RISC). The antisense strand of the duplex siRNA guides the RISC to the homologous mRNA where the RISC associated endoribonuclease cleaves the target RNA (see e.g., Zheng et al., 2004, Proc. Natl. Acad. Sci. USA 101:135; U.S. patent Publication Nos. 20040002077, 20040018176, 20030092180, 20040023390, and 20030068821).

In another embodiment the nucleic acid Abl inhibitor can be an anti-sense molecule or a ribozyme. Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarily, is not required.

A sequence “complementary” to a portion of an RNA, as discussed herein, means a sequence having sufficient complementarity to be able to hybridize with the RNA, forming a stable duplex; in the case of double-stranded antisense nucleic acids, a single strand of the duplex DNA may be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as polypeptides (e.g., for targeting host cell receptors in vivo), or agents facilitating transport across the cell membrane (see, e.g., Letsinger et al., 1989, Proc. Natl. Acad. Sci. USA 86:6553; Lemaitre et al., 1987, Proc. Natl. Acad. Sci. USA 84:648; WO 88/09810,) or the blood-brain barrier (see, e.g., WO 89/10134), hybridization-triggered cleavage agents (see, e.g., Krol et al., 1988, BioTechniques 6:958) or intercalating agents (see, e.g., Zon, 1988, Pharm. Res. 5:539). To this end, the oligonucleotide may be conjugated to another molecule, e.g., a polypeptide, hybridization triggered cross-linking agent, transport agent, or hybridization-triggered cleavage agent.

Ribozyme molecules designed to catalytically cleave target gene mRNA transcripts can also be used to prevent translation of target gene mRNA and, therefore, expression of target gene product (see, e.g., WO 90/11364; Sarver et al., 1990, Science 247:1222).

Ribozymes are enzymatic RNA molecules capable of catalyzing the specific cleavage of RNA (see Rossi, 1994, Current Biology 4:469). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. The catalytic sequence is described, e.g., in U.S. Pat. No. 5,093,246.

In one embodiment, ribozymes that cleave mRNA at site specific recognition sequences can be used to destroy target gene mRNAs. In another embodiment, the use of hammerhead ribozymes is contemplated. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, and in Haseloff and Gerlach, 1988, Nature, 334:585.

E. Transgenic Animals

A growing body of evidence has suggested that AD-related molecular factors are highly conserved in Drosophila, including the presence of amyloid precursor protein-like (Appl), γ-secretase, presenilin, Cdk5, and tau (Guo et al., 2003, Hum. Mol. Genet. 12:2669; Hellmich et al., 1994, FEBS Lett. 356:317; Muqit and Feany, 2002, Nat. Rev. Neurosci. 3:237). Thus, Drosophila may provide a powerful system for elucidating cellular mechanisms of human neurodegenerative diseases, such as AD (Chan and Bonini, 2000, Cell Death Differ. 7:1075).

The invention now provides for transgenic animals which undergo neurodegeneration that is similar to the neurodegeneration seen in AD. A transgenic animal is one engineered by humans to express an exogenous gene, i.e., one that is not expressed by the animal without human intervention. The animal may be Drosophila melanogaster and the transgene may be a gene encoding Aβ₄₂ The Aβ₄₂ may be human Aβ₄₂. In some embodiments, the transgene is ectopically expressed. In some embodiments the transgene is expressed in the compound eye. Methods of making transgenic animals which allow for the targeted controlled expression of the desired transgene have been described (see, e.g., Brand and Perrimon, 1993, Development 118:401). Thus in some embodiments the transgenic animal is comprised of the yeast trans-activator GAL4. The transgenic animal may be wild type in all respects other than the transgene or alternatively it may contain one or more mutations within its genome, e.g., within the Abl gene. In a specific embodiment the transgenic animal is a Drosophila melanogaster which expresses human Aβ₄₂. In another specific embodiment the transgenic animal is a Drosophila melanogaster which expresses human Aβ₄₂ and also contains at least one mutation in the Drosophila Abl gene. A mutation may include a substitution of at least one nucleotide in the gene sequence, the deletion of at least one nucleotide in the gene sequence, or the addition of at least one nucleotide in the gene sequence or a combination of any of these. The transgene may also contain any of the described mutations, as long as at least one activity of the protein encoded by the transgene is maintained.

The transgenic animal may be used to study neurodegenerative diseases, e.g., AD. The transgenic animal may also be used to screen for compounds that inhibit neurodegeneration and could, thus, treat AD. The transgenic animal may further be used to screen for compounds that inhibit Abl kinase activity.

Screening Assays

The invention provides for screening assays to identify Abl inhibitors. The inhibitors may be used to treat AD. The inhibitors may be used to inhibit neurodegeneration.

The assay may be a cell based assay. The cell may be of mammalian origin, e.g., human. The cell may be of insect origin, e.g., Drosophila melanogaster. In some embodiments the cell may be a neuron. The cell may be from a cell line, e.g., BG2-c2, IMR-32 or the cell may be a primary cell derived directly from an organism such as an insect or mammal.

In certain embodiments screening assay will identify agents which inhibit at least one activity associated with Abl. In one embodiment the activity inhibited by the test agent will be a kinase activity, i.e., phosphorylation of a substrate. The substrate may be any polypeptide which can be phosphorylated by Abl. The substrate may be a polypeptide endogenous to a cell used in the assay or alternatively it may be a polypeptide exogenous to a cell used in the assay, e.g., engineered by the human hand. Phosphorylation can be determined using an anti-phospho-tyrosine antibody in a Western blot or an immunoprecipitation assay, or a combination of both.

Because Abl can inhibit the cellular translocation of Cdk5, another cellular kinase, other embodiments of the invention will assay for the ability of a test agent to inhibit translocation of Cdk5. The assay can be used to screen for agents which inhibit translocation of Cdk5 within a cell, e.g., from the membrane to the perinuclear cytoplasm. Screening for the translocation of Cdk5 may be done using immunohistochemical techniques known in the art (see, e.g., Harlow et al., 1988, Antibodies A Laboratory Manual, Cold Spring Harbor Laboratory Publications, Cold Spring Harbor).

In yet another embodiment the assay may be used to screen for inhibitors which prevent formation of a complex comprising Abl, and any of Cdk5, Cables, Abi and p35 or a fragment thereof. Detection of the inhibition of the complex formation may be done by immuno-precipitation and Western blot.

In other embodiments, an in vitro kinase assay may be used to screen for Abl inhibitors. In vitro kinase assays are known in the art (see, e.g., Gaston et al., 2004, Exp. Hematol. 32:113). Abl can be recombinantly expressed and purified and combined in vitro with a substrate, e.g., Crk, Cbl. A test agent may be added and the phosphorylation of the substrate can be compared to the phosphorylation of the substrate under identical conditions without the test agent. A decrease in phosphorylation of the substrate would indicate that the test agent inhibits Abl.

G. Treatment Modalities

The Abl inhibitors of the invention can be administered intravenously, subcutaneously, intramuscularly, or via any mucosal surface, e.g., orally, sublingually, buccally, sublingually, nasally, rectally, vaginally, or via pulmonary route. The Abl inhibitors can be implanted within or linked to a biopolymer solid support that allows for the slow release of the inhbitor to the desired site.

The dose of the Abl inhibitor will vary depending on the subject and upon the particular route of administration used. Dosages can range from 0.1 to 100,000 μg/kg body weight. In one embodiment, the dosing range is 0.1-1,000 μg/kg body weight. In another embodiment the dosing range is 0.01-500 μg/kg body weight. The inhibitor can be administered continuously or at specific timed intervals. In vitro assays may be employed to determine optimal dose ranges and/or schedules for administration. In vitro assays that measure kinase activity are known in the art, e.g., immunoprecipitation and Western blot using phospho-tyrosine specific antibodies. Additionally, effective doses may be extrapolated from dose-response curves obtained from animal or insect models.

In certain embodiments the invention contemplates the administration of an nucleic acid as an Abl inhibitor. Conventional gene transfer methods may be used to introduce DNA or RNA into target cells. The precise method used to introduce the nucleic acid is not critical to the invention. For example, physical methods for the introduction of DNA into cells include microinjection and. electroporation. Chemical methods such as co-precipitation with calcium phosphate and incorporation of DNA into liposomes are also standard methods of introducing DNA into mammalian cells. DNA may be introduced using standard vectors, such as those derived from, human, murine and avian retroviruses. Other viral vectors include adeno virus and adeno associated virus (see, e.g., Gluzman et al., 1988, Viral Vectors, Cold Spring Harbor Laboratory, Cold Spring Harbor, N.Y.). Standard recombinant DNA methods are well known in the art (Ausubel et al., 1989, Current Protocols in Molecular Biology, John Wiley & Sons, New York) and viral vectors for gene therapy have been developed and successfully used clinically (Rosenberg et al., 1990, N. Engl. J. Med. 323:370).

The invention also provides a pharmaceutical composition comprising an Abl inhibitor and a pharmaceutically acceptable carrier or excipient. Examples of suitable pharmaceutical carriers are described in E. W. Martin, 1990, Remington's Pharmaceutical Sciences, 17th Ed., Mack Pub. Co., Easton, Pa. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl -p-hydroxybenzoates or sorbic acid). The preparations can also include flavoring, coloring and sweetening agents. Alternatively, the composition can be presented as a dry product for constitution with water or another suitable vehicle.

For buccal and sublingual administration the composition may take the form of tablets, lozenges or fast dissolving films according to conventional protocols.

For administration by inhalation, the compounds for use according to the present invention are conveniently delivered in the form of an aerosol spray from a pressurized pack or nebulizer (e.g., in PBS), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can be formulated for parenteral administration (i.e. intravenous or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

The pharmaceutical composition can also be formulated for rectal administration as a suppository or retention enema, e.g., containing conventional suppository bases such as cocoa butter or other glycerides.

### EXAMPLES

### Example 1

### Cell Culture, DNA Constructs and Transfection Methods

Drosophila neuronal cell line, BG2-c2 was used. It is a Drosophila melanogaster 3rd-instar larval neuronal cell line and was cultured in M3 medium (Sigma, St Louis, Mo.) plus 10% FBS (Hyclone, Logan, Utah), 10 pg/ml insulin (Sigma, St Louis, Mo.), and penicillin/streptomycin (BRL-Gibco) in 24.5° C. IMR-32 (human neuroblastoma) was obtained from ATCC (ATCC, Manassas, Va.) and propagated in MEM medium (BRL-Gibco, Carlsbad, Calif.) plus non-essential amino acids (BRL-Gibco, Carlsbad, Calif.) and 10% FBS (Hyclone, Logan, Utah).

The Drosophila constitutive expression vector used for all DNA constructs was pAc5.1 (Invitrogen, Carlsbad, Calif.). cDNA of Abl, and Ena in pPac vector were subcloned into pAc5.1. Drosophila Cdk5 was obtained from Drosophila EST LD01910 (GenBank Accession Number) and subcloned into pAc5.1 vector by digesting with restriction enzymes Not1 and Xba1. Dp35 was obtained from EST HL 05519 (GenBank Accession Number) and subsequently cloned into an HA-tagged pAc5.1 vector. Cdk5Y15F mutant was constructed by PCR-based mutagenesis. Transient transfection of neuronal cells was performed using the liposome-based method with 1˜2 μg DNA/105 cells. BG2-c2 cells were transfected with dsRNA (20 μg/2×106 cells) two days before drug treatments for another day.

### Example 2

### Cell Viability Assay

The modified MTT assay (Sigma, St Louis, Mo.) was used to quantify neuronal cell viability (Lee et al., 2000, Nature 405:360). The intensities were measured using an optical density reader (SpectroMAXplus, Molecular Devices, Sunnyvale, Calif.) at 570 nm (background: 630 nm) and proportionally represented the viable cell numbers.

### Example 3

### Immunoprecipitation and Western Blot Analysis

Cell lysate was produced in lysis buffer or extract buffer (Juang and Hoffmann,1999, Oncogene 18:5138) for Cdk5 immunoprecipitation. Proteins were analyzed by direct Western blotting (30 μg/lane) or blotting after immunoprecipitation (1˜2 mg/immunoprecipitation). Immunoprecipitates were collected by binding to protein G PLUS/Protein A-Agarose (Oncogene Research Products, Nottingham UK). The antibodies used are listed below: anti-Cdk5 antibody (clone DC17), (Upstate, Charlottesville, Va.); sc-750, (Santa Cruz Biotechnology, Inc., Santa Cruz Calif.), anti-Cdk5-pY15 antibody (sc-1 2919),(Santa Cruz Biotechnology, Inc., Santa Cruz C), anti-HA antibody (BAbCO, Berkely, Calif.), anti-p35 antibody (sc-820), (Santa Cruz Biotechnology, Inc., Santa Cruz Calif.), and affinity-purified Ena polyclonal antibody (generated by Dr. F. M. Hoffmann' Lab) (McArdle Laboratory for Cancer Research and Laboratory of Genetics, University of Wisconsin-Madison) ECL detection reagent (NEN, Boston, Mass.) was used to detect the immunoreactive proteins.

### Example 4

### In vitro Cdk5 Kinase Assay

A kinase assay was performed by washing immunoprecipitates three times with kinase reaction buffer (50 mM HEPES [pH 7.0], 10 mM MgCl2, and 1 mM DTT). The beads with target proteins were incubated with kinase reaction buffer containing 2 μg of substrate (histone H1)(Calbiochem, San Diego, Calif.) and 10 μCi of ³³Pγ-ATP in a final volume of 40 μl at 30° C. for 30 minutes.

### Example 5

### Double Stranded RNA Synthesis

DNA fragments including both sense and antisense coding sequences for Drosophila abl (729 bp, CDS: 241˜969), EGFP (full-length, 765 bp, Invitrogen, Carlsbad, Calif.), and Dp35 (720 bp, CDS: 1-720) were amplified by PCR. PCR products were subcloned into the vector (all cut with BamH1 and blunt-end products were produced), pBluescript, cut by EcoRV, (Strategen, Wakefield, Wash.) and subsequently used as a template for in vitro transcription by T3/T7 MEGAscript kit (Ambion, Austin, Tex.). Single stranded RNA was generated and annealed with compensatory strands in 65° C. for 30 minutes, followed by 94° C., for 5 minutes and 72° C., for 10 minutes. Double stranded RNA products were confirmed as a single band by electrophoresis and stored at −20° C.

### Example 6

### Immunohistochemistry

Neuronal cells cultured on coverslips (coated by poly-L-lysine) (Biochrom AG, Berlin, Germany) were fixed, permeabilized, and blocked as previous described (Gertler et al., 1995, Genes Dev. 9:521). Primary antibodies (anti-Cdk5, 2 μg/ml; anti-HA, 1:500) diluted in 3% BSA-PBS were incubated with coverslips overnight at 4° C. Cells were washed in PBS and exposed to FITC or TRITC-conjugated secondary antibodies (both affinity-purified goat anti-mouse lgG, 1:2000, (Jackson ImmunoResearch Laboratories, Inc, West Grove, Pa.) for one hour at room temperature. After extensive washing, coverslips were mounted in Gel/Mount (Biomeda, Foster City, Calif.) and observed by Leica confocal microscopy. The TUNEL (TdT-mediated dUTP nick end labeling) assay was carried out using the In Situ Cell Death Detection Kit™ (Roche, Nutley, N.J.). Third-instar larvae were dissected in M3 medium and eye imaginal discs were fixed in 4% paraformaldehyde/PBS for 20 minutes at room temperature and then washed three times with PBS-T (PBS/0.3% Triton X-100). The samples were incubated in the TUNEL reaction mixture (including FITC-conjugated modified nucleotides and terminal deoxynucleotidyl transferase) for one hour at 37° C. TUNEL signals were visualized directly under Leica confocal microscopy with a FITC filter.

### Example 7

### Generation of Transgenic Flies

A cDNA encoding the fragment of human amyloid precursor protein, Aβ_{42,} was amplified from a human brain cDNA library by PCR with primer 5′-AAGATGGATG CAGAATTCCG ACATGACTCA GGA-3′ (SEQ ID NO: 1) and 5′TTAATGATGA TGATGATGAT GCGCTATGAC AACACCGCCCAC CATGAGTC CAAT-3′ (SEQ ID NO: 2). cDNAs of Aβ₄₂ and Dp35 were separately sequenced and subcloned into the pUAST vector which was used to microinject to generate transgenic flies. Embryos of the A23/+ genotype fly were microinjected and 10 transgenic lines were obtained by standard methods (Rubin and Spradling, 1982, Science 218:348). The transgenic lines which showed obvious red-eye phenotypes were selected. Standard genetic balancers and chromosomes were used as described (Lindsley and Zimm, 1992, The genome of Drosophila melanogaster (San Diego: Academic Press). Abl-1 and Df(3L)stj7, Ki strand flies were provided from Bloomington Drosophila Stock Center (Bloomington, Ind.) and crossed with transgenic flies to obtain Abl-/- background flies with ectopically expressed proteins. Double transgenic flies which expressed both Aβ₄₂ and Dp35-HA were also generated by sequential crossing. All ectopically expressed Aβ₄₂ and Dp35-HA were expressed under the control of gmr-GAL4 in fly eyes.

### Example 8

### Aβ₄₂ Activates Abl Kinase and Induces Drosophila Neuronal Death

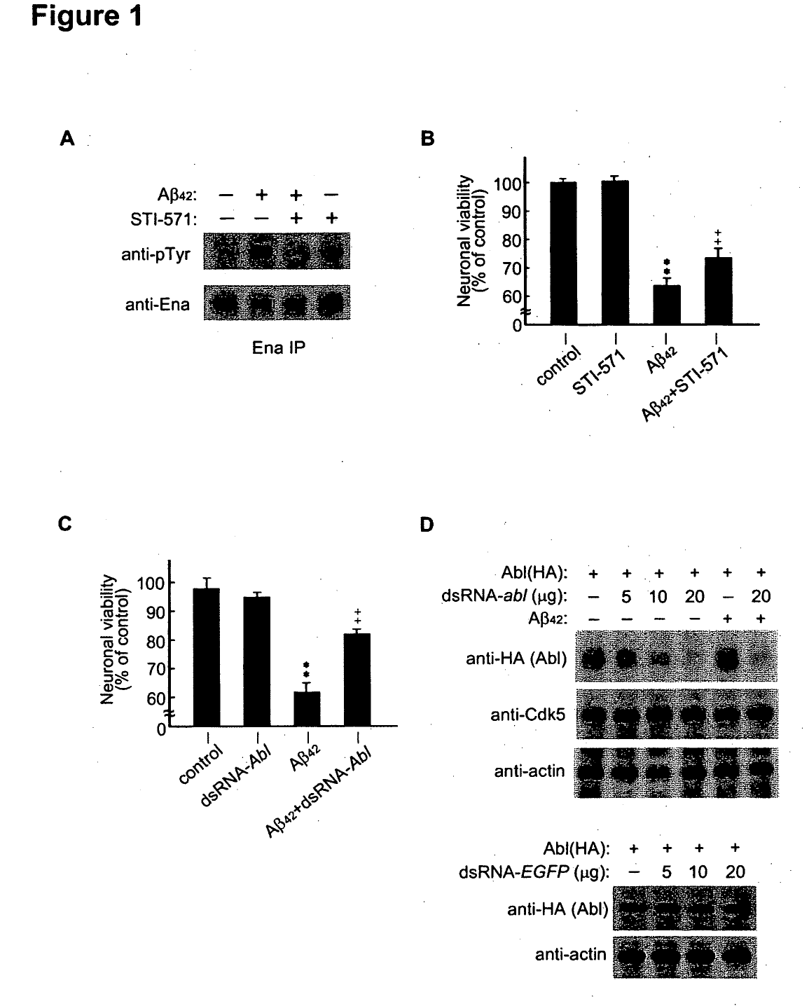

To determine whether Abl kinase activity is modulated by Aβ₄₂ in neurons, Drosophila neuronal cells, BG2-c2 (Ui-Tei et al,1996, Neurosci. Lett. 203:191) were treated with Aβ₄₂ (2 μM) and Abl kinase activity was indirectly evaluated by monitoring the tyrosine-phosphorylation level of Ena, a kinase substrate that was previously found to be primarily phosphorylated by Abl in Drosophila cells (Gertler et al., 1995, Genes Dev. 9:521; Juang and Hoffmann, 1999, Oncogene 18:5138). Notably, Ena phosphorylation levels were elevated by Aβ₄₂ but reduced by STI571 (Gleevec, Imatinib) (20 μM) (FIG. 1A). The result shows Abl kinase activity was stimulated by Aβ₄₂.

To determine whether activated Abl kinase was necessary for Aβ₄₂ induced neuronal death, Abl kinase activity was suppressed by STI571 in Aβ₄₂ treated neurons and cell survival was monitored using the assay described above. FIG. 1B shows that the treatment with STI571 rescued Aβ₄₂-induced neuronal death. Consistently, reduction of Abl protein expression by dsRNA also prevented Aβ₄₂-induced neuronal death (FIG. 1C). The dsRNA-abl markedly suppressed the Abl expression in a dosage-dependent manner and was effective in cells treated with Aβ₄₂ (FIG. 1D). No detectable viability change was observed after treatment with control dsRNA-EGFP. These data suggest that Abl kinase is involved in the Aβ₄₂-triggered Drosophila neuronal death.

### Example 9

### Activated Abl Kinase Allows for Abl-Cdk5 Interaction and Cdk5 Activation

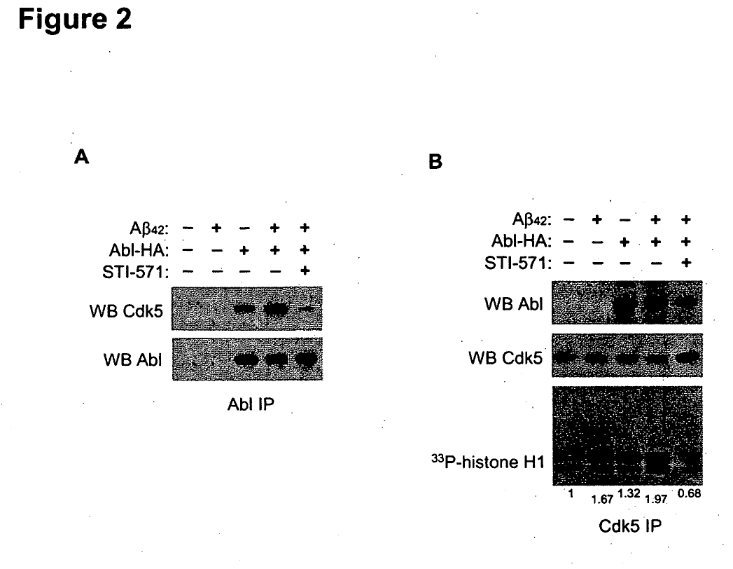

To determine if Abl was an upstream regulator of cdk5 activation Aβ₄₂-triggered Abl activation was investigated to determine if it could affect the Abl-cdk5 physical association. The endogenous Cdk5 was co-immunoprecipitated with exogenous HA-tagged Abl in BG2-c2 cells to determine the physical interaction between Abl and Cdk5. The complex of Abl and Cdk5 was enhanced upon Aβ₄₂ treatment (lane 4 in FIGS. 2A and 2B). Moreover, suppression of Abl kinase activity with STI571 dramatically abolished the Abl-Cdk5 association in cells (lane 5 in FIGS. 2A and 2B). These results suggest that the Abl-Cdk5 interaction is correlated to the Aβ₄₂-initiated Abl kinase activity.

Whether Cdk5 was activated in the Abl-Cdk5 complex was also investigated. Cdk5 was immunoprecipitated from the cell lysate for an vitro kinase assay, using histone H1 as a substrate (Connell-Crowley et al., 2000, Curr. Biol. 10:599). Similar to results reported for mammalian cells (Alvarez et al., 2001, Exp. Cell Res. 264:266), Drosophila Cdk5 kinase activity was elevated following Aβ₄₂ treatment as compared to controls (FIG. 2B, bottom panel). The elevated Cdk5 kinase activity was consonant with the activated Abl kinase because suppression of Abl kinase activity with STI571 not only dissociated the Abl-Cdk5 interaction, but also sharply diminished Cdk5 kinase activity (FIG. 2B). Moreover, other Abl kinase regulating agents including doxorubicin (DX) and erbstatin analog (EA) were used to activate or suppress Abl kinase activity, respectively, and confirmed that Abl kinase activity was required for modulation of Cdk5 kinase. Taken together, these results demonstrate that Abl kinase activity is required for Abl-Cdk5 interaction and Cdk5 activation in Aβ₄₂-triggered neurodegeneration.

### Example 10

### Genetic and Cell-Based Assays Show Abl is Essential in Activating Cdk5 Kinase for Aβ₄₂-Triggered Neurodegeneration

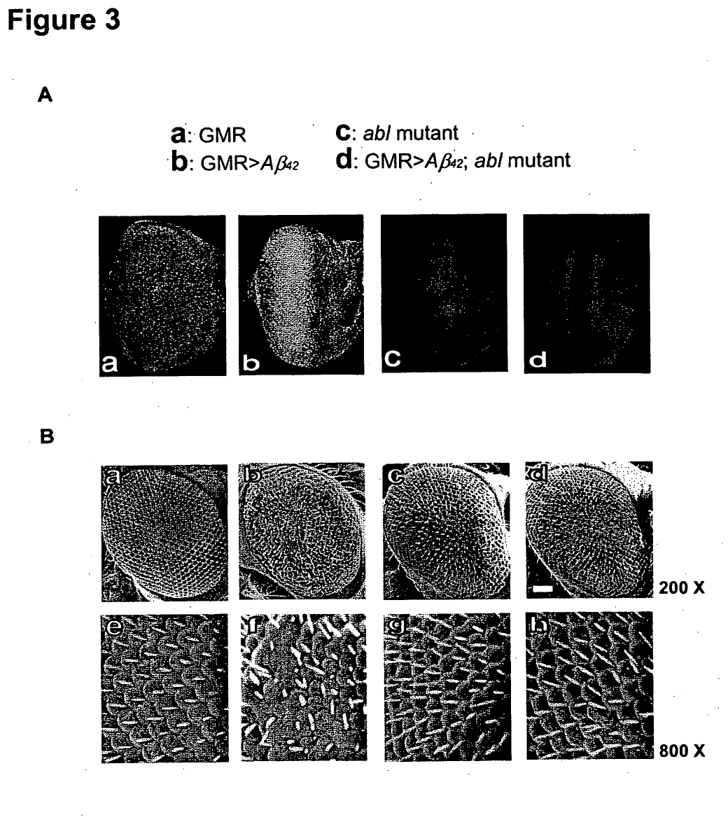

To demonstate that Abl is essential in activating Cdk5 kinase for Aβ₄₂-triggered neurodegeneration in vivo, a transgenic fly model which ectopically expresses Aβ₄₂ in the developing compound eye was established. By crossing the UAS-Aβ₄₂transgenic flies with the eye-specific gmr-GAL4 driver in either wild-type or Abl mutant backgrounds (Df(3L)stj7, Ki/abl[1]) (Bennett and Hoffmann, 1992, Development 116:953), the role of Abl in neurodegeneration based on the severity of eye phenotypes in larval and adult stages was tested. Neurodegenerative phenotypes were determined using TUNEL staining to reveal the neuronal apoptosis (see, e.g., McPhie et al., 2003, J. Neurosci. 23:6914). In the wild-type background, expression of Aβ₄₂ behind the morphogenetic furrow in the 3rd-instar larval eye discs resulted in severe apoptosis in posterior region of the eye disc compared to gmr-GAL4 control (compare FIGS. 3A-b and 3A-a). Notably, an Abl mutant led to suppression of Aβ₄₂-induced apoptosis (FIG. 3A). The Aβ₄₂-induced apoptotic cells in the larval eye discs subsequently led to facet disorder (fused ommatidium) and bristle lost in adult compound eyes (FIG. 3B). Similarly, the Aβ₄₂-induced adult compound eye phenotypes were suppressed by mutation of Abl (FIG. 3B). These genetic results suggest that Abl is an important factor contributing to Aβ₄₂ induced Drosophila neurodegeneration.

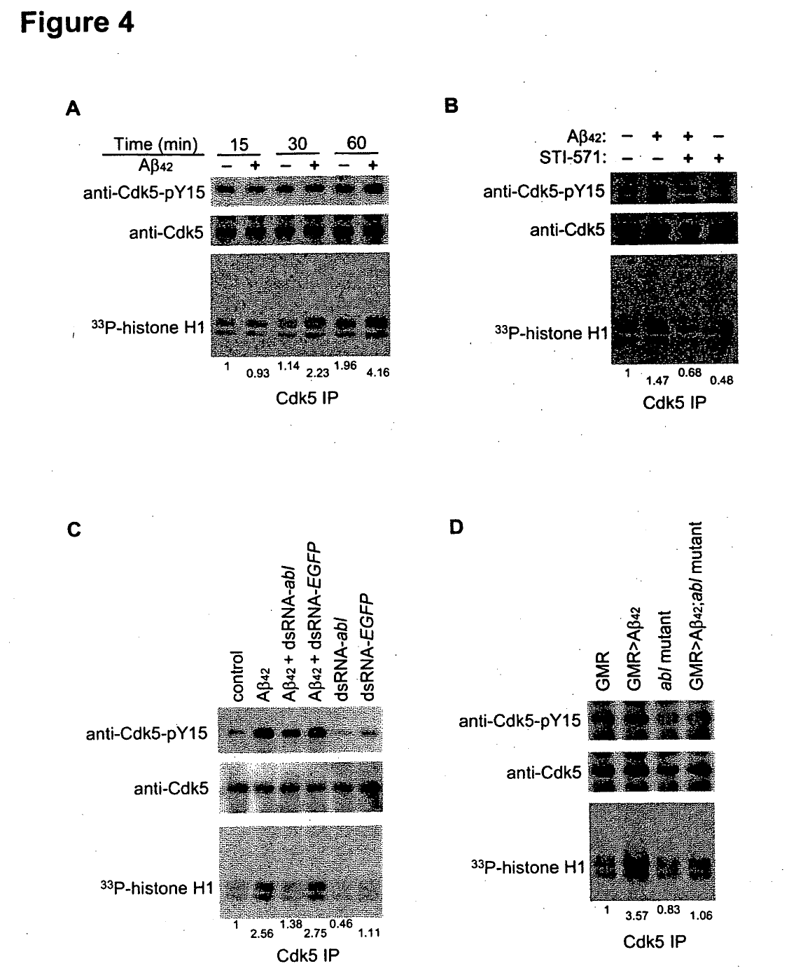

To understand the role of Drosophila Cdk5 in neurodegeneration, the phosphorylation of Cdk5-Y15 was investigated to determine if it was correlated with the pathological activation of Cdk5 in neuronal cells. Cdk5-Y15 phosphorylation (Cdk5-pY15) levels in BG2-c2 cells were examined over time following treatment with 2 μM Aβ₄₂. The presence of Cdk5-pYll15 in the immunoprecipitates was shown by immunoblotting with an anti-phospho antibody specific for Cdk5-pY15. Within 60 minutes, Aβ₄₂ caused an elevation of Cdk5-pY15, which was coincident with an increase in Cdk5 kinase activity (FIG. 4A). A kinase-inactive Cdk5 (Cdk5-Y15F) as transfected into the Aβ₄₂-stimulated neuronal cells and demonstrated that the Cdk5-pY15 signal and neuronal death were diminished. These results show that Abl is a Cdk5-Y15 kinase which triggers Cdk5 kinase activity in Aβ₄₂-stimulated neurons. To confirm this result, Abl kinase activity was suppressed with STI571 in Aβ₄₂-treated BG2-c2 cells. The result showed that both the Cdk5-pY15 and kinase activity were diminished (FIG. 4B). Moreover, dsRNA-abl (20 μg/2×10⁶ cells) was transfected into BG2-c2 cells to reduce Abl expression two days before Aβ₄₂ treatment. Consistent with the results obtained with STI571, these cells exhibited a reduction of Cdk5-pY15 and kinase activity (FIG. 4C), confirming Abl is crucial for Aβ₄₂-induced Cdk5 activation.

This finding was further substantiated in vivo by immunoprecipitating endogenous Cdk5 from wild type and Abl mutant adult fly heads to compare their Cdk5-pY15 levels and Cdk5 kinase activities. The Cdk5-pY15 signal and kinase activity were both elevated in flies overexpressing Aβ₄₂, as compared to control flies (FIG. 4D, lanes 1 and 2). In contrast, overexpressing Aβ₄₂ in abl mutant flies resulted in the reduction of Cdk5-pY15 signal and Cdk5 kinase activity (FIG. 4D, lanes 3 and 4). In summary, Abl is involved in Aβ₄₂-induced neurodegeneration via the regulation of Cdk5.

### Example 11

### p35 is not Cleaved into p25 in the Drosophila Neurodegeneration Model

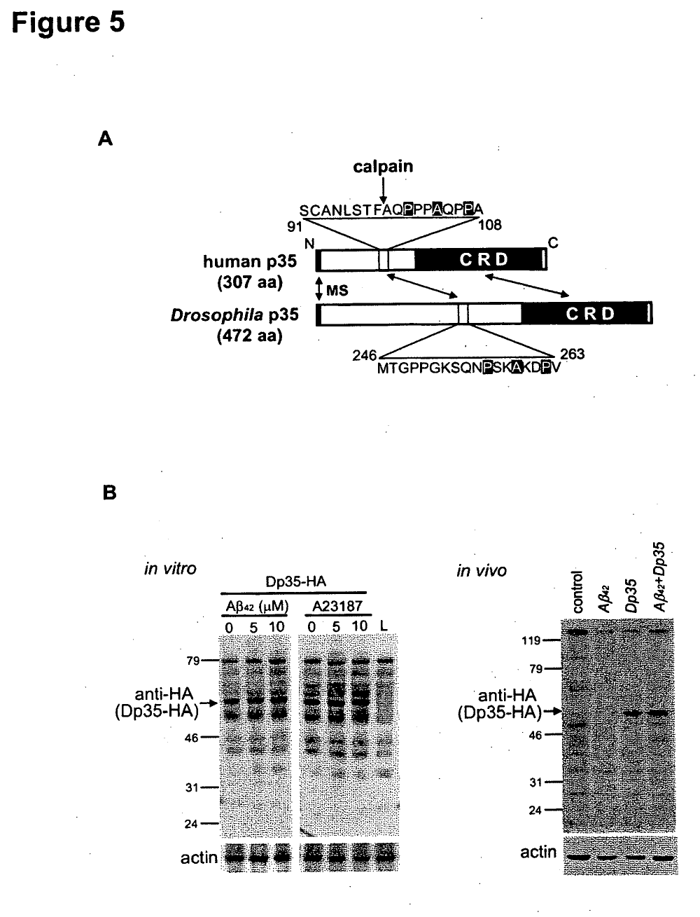

Like Abl, p35 is conserved in Drosophila and functions to modulate Cdk5 activity during neurite outgrowth (Connell-Crowley et al., 2000, Curr. Biol. 10:599). In the AD brain, mammalian p35 is cleaved into p25 by calpain to deregulate Cdk5 (Lee et al., 2000, Nature 405:360). Although a putative calpain is also present in Drosophila (Jekely and Friedrich, 1999, J. Biol. Chem. 274:23893), the consensus calpain cleavage site is absent in Drosophila p35 (Dp35) (FIG. 5A). Therefore, it was unclear whether Dp35 would be converted into p25 upon Aβ₄₂ stimulation. To investigate this possibility, carboxyl-terminal HA-tagged Dp35 was transfected into BG2-c2 cells before treating them with Aβ₄₂or A23187/Ca²⁺ (calcium ionophore). Intriguingly, no noticeable cleavage or reduction of Dp35 protein levels in the lysates were observed (FIG. 5B, left panel). To analyze the Dp35 cleavage in vivo, transgenic flies co-expressing Dp35 and Aβ₄₂ in the developing eyes were generated using a gmr-GAL4 driver. Consistent with the in vitro result, no visible cleavage or reduction of the Dp35 protein in the Aβ₄₂ transgenic flies were observed (FIG. 5B, right panel). These results show that Dp25 or other truncated forms of Dp35 protein is not required for the Aβ₄₂-induced Drosophila neurodegeneration. However, the absence of Dp35 cleavage in neurodegenerative flies does not necessarily imply that p35 is dispensable for the regulation of Cdk5 in Drosophila neurodegeneration.

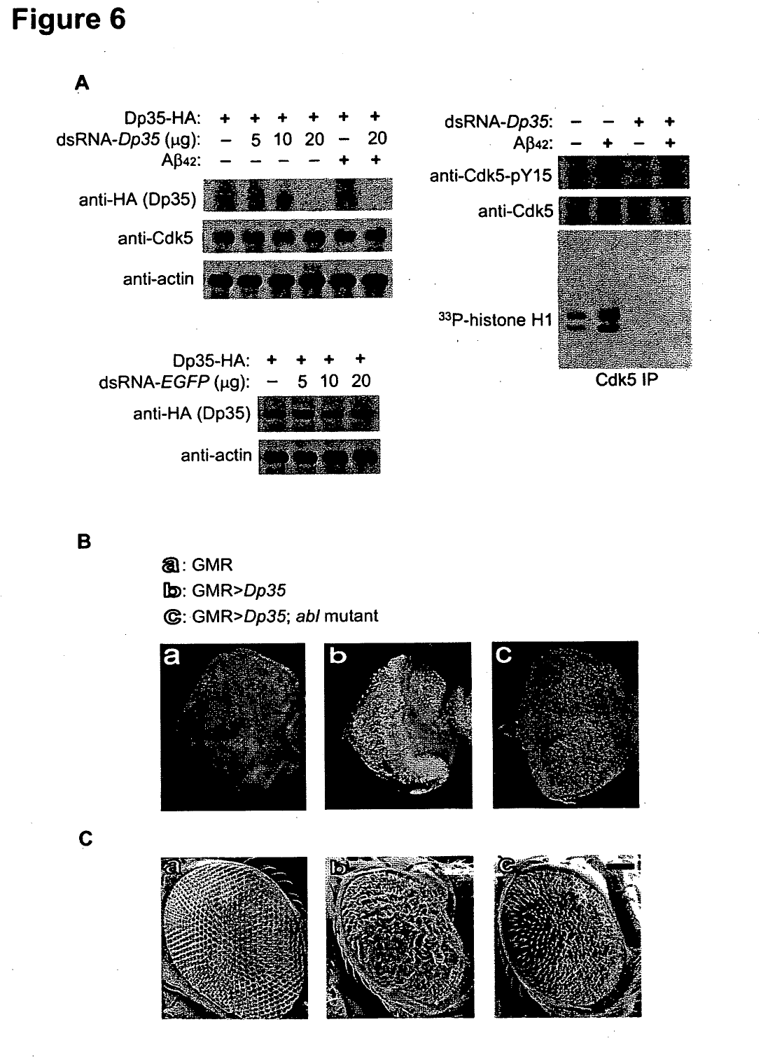

To explore whether Dp35 is necessary for Aβ₄₂-induced Cdk5 activation in Drosophila, dsRNA-Dp35 was transfected into neuronal cells prior to stimulating cells with Aβ₄₂. The Dp35 protein expression levels were markedly suppressed by dsRNA-Dp35 in a dose-dependent manner, while no off-target, non-specific effect was observed (FIG. 6A, left panel). Although Cdk5-Y15 was still phosphorylated in response to Aβ₄₂, the Cdk5 kinase activity was virtually abolished after Dp35 was depleted from cells (FIG. 6A, right panel). This result demonstrates that, while Dp35 is not essential for Abl phosphorylation of Cdk5-Y15, the induction of Cdk5 by Abl still requires the involvement of p35.

To address the cooperative role of Abl and p35 in neurodegeneration, a genetic interaction assay was conducted. Overexpression of Dp35 in the developing photoreceptor cells by a gmr-GAL4 driver resulted in apparent apoptosis in the 3rd-instar larval eye discs (FIG. 6B-b) and severe adult rough eye phenotypes (FIG. 6C-b). Consistent with the results from Aβ₄₂-induced eye phenotypes, the Dp35-induced neuronal apoptosis in the eye disc and compound eyes were suppressed by the Abl mutant (FIGS. 6B and 6C). Because overexpression of Dp35 was shown to induce Cdk5 hyperactivation (Connell-Crowley et al, 2000, Curr. Biol. 10:599), the finding that the Abl mutant suppressed Dp35-induced neurodegeneration further substantiates the role Abl plays in conjunction with Cdk5 in neurodegeneration.

### Example 12

### Cdk5-Y15 Phosphorylation by Abl is Required for Aβ₄₂-Induced Cdk5 Subcellular Translocation

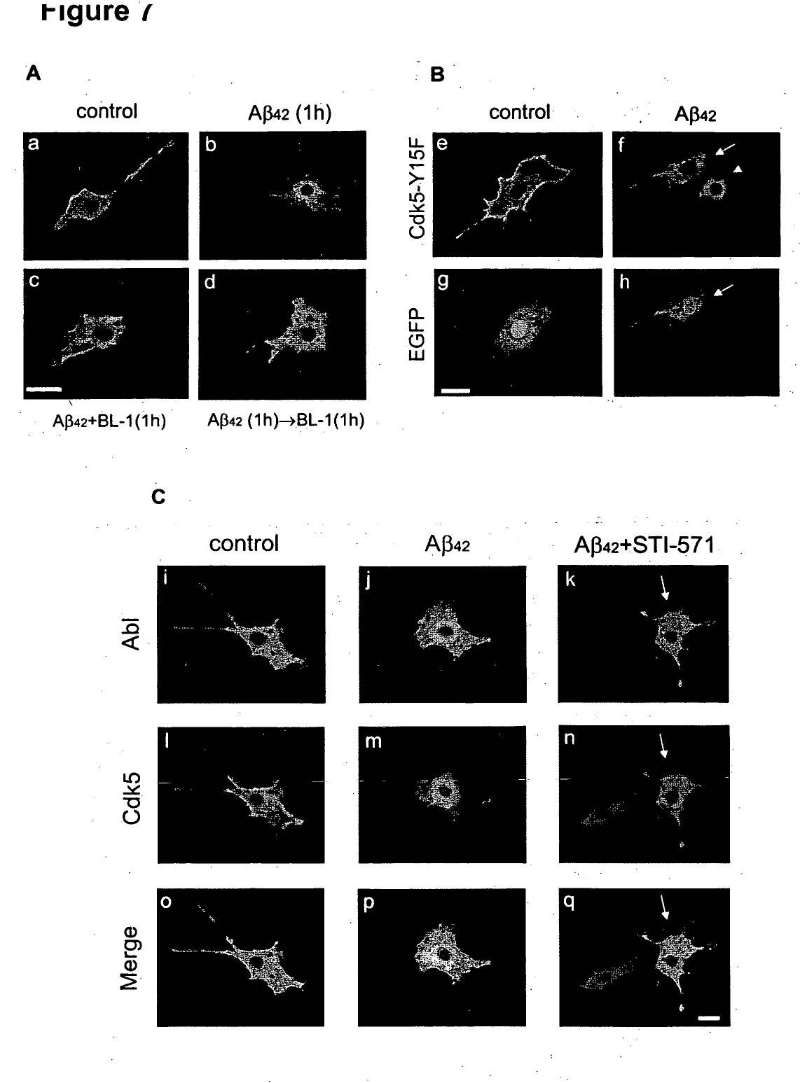

It was postulated that the amino-terminal myristoylation sequence of mammalian p35 is the key domain for anchoring the Cdk5-p35 complex to the cell membrane. Removal of the p35 amino-terminus, producing p25, would sever the complex's association with the cell membrane and thus translocalize Cdk5 to cytosol (Patrick et al., 1999, J. Biol. Chem. 273:24057). Intriguingly, although the cleavage of p35 into p25 may be absent in drosophila neurodegenerative neurons, the myristoylation site is conserved in Dp35 (FIG. 5A). The unique sequence feature of Dp35 implies that Drosophila Cdk5-p35 complex may be resident on the cell membrane and cannot be shuttled to the cytosol. To explore this, endogenous Cdk5 protein localization was examined by immunostaining. Without the stimulus of Aβ₄₂, the Cdk5 was primarily expressed on the cell membrane with lesser amounts found in the cytosolic region (FIG. 7A, panel a). This finding is similar to the result observed in mammalian cells (Matsushita et al., 1995, Neuroreport 6:1267). Unexpectedly, one hour after a 2 μM Aβ₄₂stimulus, Cdk5 was markedly translocalized from cell membrane to the perinuclear cytoplasm (FIG. 7A, panel b), which suggests a p25-independent mechanism for Cdk5 translocalization. By treating cells with Aβ₄₂ simultaneously with BL-1 (Cdk5 kinase inhibitor) to block the Cdk5 activation, the translocalization of Cdk5 to the perinuclear cytoplasm was abrogated (FIG. 7A, panel c). This result indicated a kinase-dependent regulation of Cdk5 translocalization. Moreover, we discovered that the Cdk5 protein which had been translocalized to the perinuclear cytoplasm could be shuttled back to plasma membrane by suppressing Cdk5 kinase activity one hour after Aβ₄₂ treatment (FIG. 7A, panel d). These results suggest that Drosophila Cdk5, in response to Aβ₄₂ stimulus, can be reversibly shuttled between the cell membrane and perinuclear cytoplasm by a p25-independent mechanism.

Since phosphorylation of Cdk5-Y15 correlates with Cdk5 activity, whether the Cdk5-pY15 is required for directing Cdk5 subcellular localization was investigated. Enhanced green fluorescent protein (EGFP) was co-transfected with the Cdk5-Y15F mutation plasmid to label the neurons with exogenous Cdk5-Y15F protein expression. Without the Aβ₄₂ stimulus, Cdk5-Y15F was primarily localized to the cell membrane similar to the result seen with the wild-type Cdk5 (FIG. 7B, panel e). However, treatment of Aβ₄₂ did not noticeably affect the subcellular localization of Cdk5-Y15F (FIG. 7B, panel f, arrow), in contrast to the untransfected cells (FIG. 7B, panel f, arrowhead). Thus, the translocation of Cdk5 in response to A(342 stimulus may be modulated by the Abl phosphorylation of Cdk5-Y15. To test this idea, Abl kinase activity was suppressed with STI571 before stimulating cells with Aβ₄₂ and immunostained both the Abl-HA and the endogenous Cdk5 proteins in the cells. Abl and Cdk5 were colocalized to the plasma membrane and cytoplasm in control cells (FIG. 7C, panels i, l, and o). Upon stimulation with Aβ₄₂, Cdk5 colocalized with Abl to the perinuclear cytoplasm (FIG. 7C, panels j, m, and p). However, such protein translocalization was markedly suppressed when Abl activity was blocked (FIG. 7C, panels k, n, and q, arrow). dsRNA-abl was transfected into neuronal cells and found a result similar to that seen with STI571. These results accord well with the role of Abl induced modulation of Cdk5 and indicate a strong link between Abl regulation of Cdk5 and neurodegeneration.

### Example 13

### Aβ₄₂-Triggered Human p35 Cleavage into p25 is Insufficient to Initiate Cdk5 Kinase Activity in Cells Deficient of Abl Kinase Activity

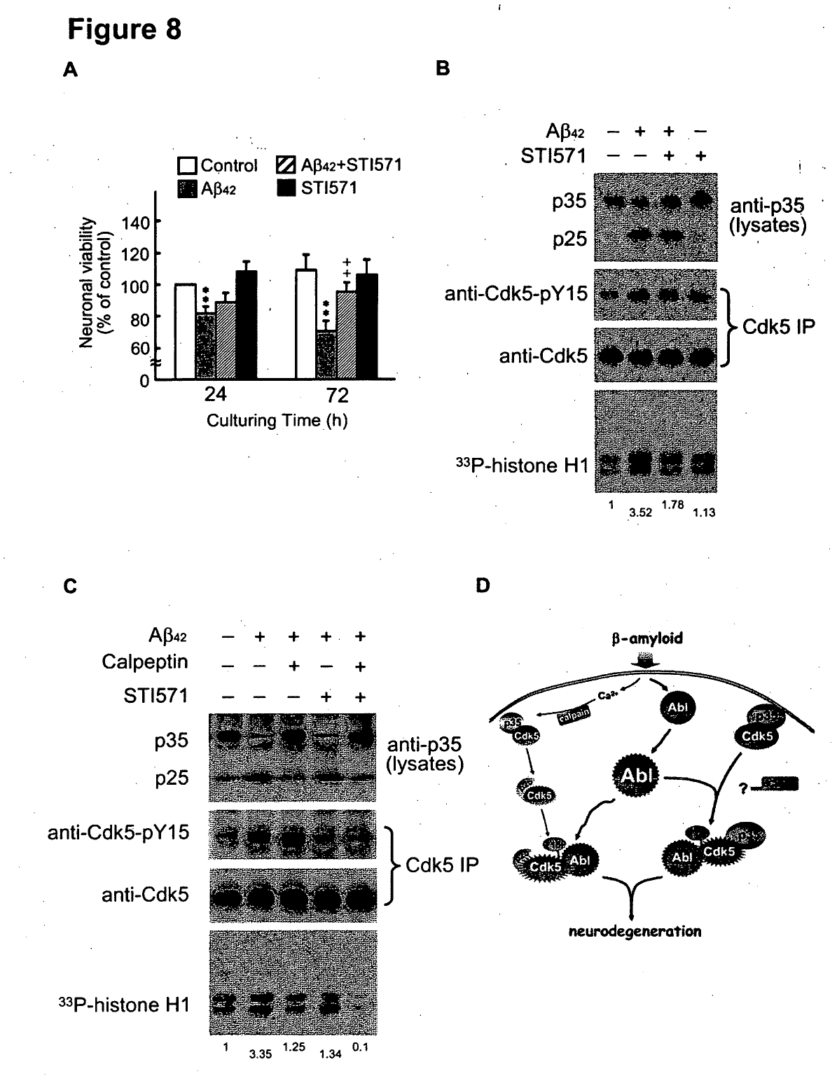

To determine if the role of Abl in Drosophila neurodegeneration is conserved in mammals, Abl kinase activity in Aβ₄₂-induced mammalian neuronal apoptosis was investigated. Abl kinase was suppressed with STI571 in human IMR-32 cells 24 hours prior to treatment with Aβ₄₂, and neuronal viability was determined. In accord with the result in Drosophila model system, the inhibition of Abl kinase activity rescued the neuronal death in IMR-32 cells (FIG. 8A). Formation of p25 has been considered a causative agent that contributes to the deregulation of Cdk5 in human AD (Lee et al., 2000, Nature 405:360; Patrick et al., 1999, Nature 402:615). However, results of the Drosophila neurodegeneration model suggest that Abl may be critical in regulating Cdk5. Thus, it was important to determine whether the conversion of p35 into p25 is sufficient to deregulate Cdk5 activity while Abl activation is inhibited. After pretreating with STI571 for 24 hours to block Abl kinase activity, IMR-32 cells were incubated with Aβ₄₂to induce the cleavage of human p35. The cells were then assayed for Cdk5 kinase activity and Y-15 phosphorylation. The results obtained were in accord with previously reported findings and showed that Aβ₄₂triggered the cleavage of p35 into p25, increased Cdk5-pY15 and increased kinase activity (FIG. 8B, lane 2) (Alvarez et al., 2001, Exp. Cell Res. 264:266). However, in the presence of STI571, both the Cdk5-pY15 and kinase activation were abolished, though the formation of p25 was not obviously affected (FIG. 8B, lane 3). This result indicates that the cleavage of p35 into p25 is insufficient to activate Cdk5 while Abl kinase activity is suppressed.

To determine whether p25 functions cooperatively with Abl to assist the maximal activation of Cdk5 kinase (because the p25-Cdk5 complex is more stable than that of p35-Cdk5 (Patrick et al, 1998, J. Biol. Chem. 273:24057; Tarricone et al, 2001, Mol. Cell 8:657)), a calpain inhibitor (Calpeptin) (Calbiochem San Diego, Calif.) was used to inhibit the cleavage of p35 in Aβ₄₂-treated IMR-32 cells. Interestingly, the blockade of p35 cleavage did not diminish Cdk5-pY15 but the Cdk5 activity was considerably reduced (FIG. 8C, compare lanes 2 and 3), indicating that the formation of p25 is indeed important for the Cdk5 activation whereas the phosphorylation of Cdk5-Y15 is independent of p35 cleavage. In addition, we also found that the co-treatment of cells with Calpeptin and STI571 further reduced the Cdk5 kinase activity (FIG. 8C, compare lane 5), confirming that Abl and p25 function cooperatively in deregulating Cdk5 in mammalian cells. Taken together, this data demonstrates that Abl is a critical component, which mediates Cdk5-pY15 and Cdk5 activation in Aβ₄₂-triggered neurodegeneration, and the cleavage of p35 into p25 functions to stabilize the Abl-Cdk5 complex for the maximal induction of Cdk5 activity.

All references cited herein are incorporated herein by reference in their entirety. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

1. A method of treating a subject having a neurodegenerative disease comprising administering to the subject, an agent which inhibits Abl kinase activity.

2. The method of claim 1, wherein the neurodegenerative disease is Alheimer's disease.

3. The method of claim 1, wherein the agent is a polypeptide, a nucleic acid, or a small molecule.

4. The method of claim 3, wherein the nucleic acid is an RNA molecule.

5. The method of claim 4, wherein the RNA molecule is siRNA molecule.

6. The method of claim 4, wherein the RNA molecule is ds-RNA Abl.

7. The method of claim 3, wherein the small molecule is STI571.

8. The method of claim 3, wherein the agent is an erbstatin analog.

9. The method of claim 1, wherein the subject is a human.

10. The method of claim 1, wherein the Abl kinase activity inhibited is phosphorylation of a substrate.

11. The method of claim 10, wherein the substrate is another kinase.

12. The method of claim 11, wherein the substrate is a cyclin dependent kinase.

13. The method of claim 12, wherein the cyclin dependent kinase is cyclin dependent kinase 5 (Cdk5).

14. A method of inhibiting degeneration of a neuron comprising contacting the neuron with an agent which inhibits Abl kinase activity.

15. The method of claim 14, wherein the neuron is a mammalian neuron.

16. The method of claim 15, wherein the mammalian neuron is a human neuron.

17. The method of claim 14, wherein the neuron is a Drosophila melanogaster neuron.

18. The method of claim 14, wherein the agent is a polypeptide, a nucleic acid, or a small molecule.

19. The method of claim 18, wherein the nucleic acid is an RNA molecule.

20. The method of claim 19, wherein the RNA molecule is siRNA molecule.

21. The method of claim 19, wherein the RNA molecule is ds-RNA Abl.

22. The method of claim 14, wherein the small molecule is STI571.

23. The method of claim 14, wherein the agent is an erbstatin analog.

24. A method of inhibiting formation of a complex comprising Abl, Cdk5, Cables, Abi and p35 or a fragment thereof, in a neuron comprising, contacting the neuron with an agent which inhibits Abl kinase activity.

25. The method of claim 24, wherein the neuron is a mammalian neuron.

26. The method of claim 25, wherein the mammalian neuron is a human neuron.

27. The method of claim 24, wherein the neuron is a Drosophila melanogaster neuron.

28. The method of claim 24, wherein the agent is a polypeptide, a nucleic acid, or a small molecule.

29. The method of claim 28, wherein the nucleic acid is an RNA molecule.

30. The method of claim 29, wherein the RNA molecule is siRNA molecule.

31. The method of claim 29, wherein the RNA molecule is ds-RNA Abl.

32. The method of claim 28, wherein the small molecule is STI571.

33. The method of claim 28, wherein the agent is an erbstatin analog.

34. The method of claim 24, wherein the Abl kinase activity inhibited is phosphorylation of a substrate.

35. The method of claim 34, wherein the substrate is another kinase.

36. The method of claim 35, wherein the substrate is a cyclin dependent kinase.

37. The method of claim 36, wherein the cyclin dependent kinase is cyclin dependent kinase 5.

38. A method of inhibiting Cdk5 activity in a cell comprising contacting the cell with an agent which inhibits Abl kinase activity thereby inhibiting Cdk5 activity.

39. The method of claim 38, wherein the agent is a polypeptide, a nucleic acid, or a small molecule.

40. The method of claim 39, wherein the nucleic acid is an RNA molecule.

41. The method of claim 40, wherein the RNA molecule is siRNA molecule.

42. The method of claim 41, wherein the RNA molecule is ds-RNA Abl.

43. The method of claim 39, wherein the small molecule is STI571.

44. The method of claim 39, wherein the agent is an erbstatin analog.

45. The method of claim 38, wherein the Abl kinase activity inhibited is phosphorylation of a substrate.

46. A method of screening for an agent which treats a neurodegenerative disease comprising
a) contacting a cell with the agent; and
b) detecting at least one of Abl kinase activity in the cell and changes in Cdk5 subcellular localization
where a decrease in Abl kinase activity in the cell or a change in Cdk5 subcellular localization contacted with the agent compared to a cell not contacted with the agent indicates that the agent can be used to treat a neurodegenerative disease.

47. The method of claim 46, wherein the neurodegenerative disease is Alheimer's disease.

48. The method of claim 46, wherein the agent is a polypeptide, a nucleic acid, or a small molecule.

49. The method of claim 46, wherein the Abl kinase activity is phosphorylation of a substrate.

50. The method of claim 46, wherein the cell is a neuron.

51. The method of claim 50, wherein the neuron is a mammalian neuron.

52. The method of claim 51, wherein the mammalian neuron is a human neuron.

53. The method of claim 50, wherein the neuron is an insect neuron.

54. The method of claim 53, wherein the insect neuron is a drosophila melanogaster neuron.

55. A method of screening for an agent which inhibits degeneration of a neuron comprising contacting a cell with the agent and detecting at least one of the Abl kinase activity in the cell and Cdk5 protein subcellular localization, where a decrease in Abl kinase activity in the cell contacted with the agent or a change in Cdk5 protein subcellular localization compared to a cell not contacted with the agent indicates that the agent can be used to inhibit neurodegeneration.

56. The method of claim 55, wherein the agent is a polypeptide, a nucleic acid, or a small molecule.

57. The method of claim 55, wherein the Abl kinase activity is phosphorylation of a substrate.

58. The method of claim 55, wherein the cell is a neuron.

59. The method of claim 58, wherein the neuron is a mammalian neuron.

60. The method of claim 59, wherein the mammalian neuron is a human neuron.

61. The method of claim 60, wherein the neuron is an insect neuron.

62. The method of claim 61, wherein the insect neuron is a drosophila melanogaster neuron.

63. A transgenic animal comprising a transgene comprising a nucleotide sequence encoding a β amyloid protein.

64. The transgenic animal of claim 63, wherein the animal is drosophila melanogaster.

65. The transgenic animal of claim 63, wherein the β amyloid protein is Aβ₄₂.

66. The transgenic animal of claim 65, wherein the β amyloid protein is ectopically expressed.

67. The transgenic animal of claim 66, wherein the β amyloid protein is ectopically expressed in the compound eye.

68. The transgenic animal of claim 63, wherein the transgenic animal expresses a mutant form of Abl.

69. The transgenic animal of claim 68, wherein the transgenic animal expresses a wild type form of Abl.

70. The transgenic animal of claim 69, wherein the transgenic animal comprises a nucleotide sequence encoding GMR-gal 4.
